(19) United States
(12) Patent Application Publication
(10) Pub. No.: US 2017/0281149 A1
(43) Pub. Date: Oct. 5, 2017

(54) SELF-RETAINING RADIAL TISSUE RETRACTOR

(71) Applicant: OriGYN Medical Inc., Decatur, GA (US)

(72) Inventors: Christopher Rolfes, Decatur, GA (US); Gavin Sillitto, Edinburgh (GB); Kathryn McAlexander, Atlanta, GA (US); Ashley Saienni, Atlanta, GA (US); Pamela Collier, Acworth, GA (US); Robert Steed, Sandy Springs, GA (US)

(21) Appl. No.: 15/463,995

(22) Filed: Mar. 20, 2017

Related U.S. Application Data
(60) Provisional application No. 62/311,245, filed on Mar. 21, 2016.

(51) Int. Cl. A61B 17/02

(52) U.S. Cl. CPC A61B 17/0293; A61B 2017/00862

(57) ABSTRACT

Disclosed is a self-retaining retractor configured for insertion into an orifice. The self-retaining retractor may include a flexible structure made of, for example, one or more of an elastic polymer sheet and an elastic polymer mesh. Further, the self-retaining retractor may include a flexible or shape memory material, such as, for example, nitinol, polymer or elastomer, incorporated in the flexible structure. Further, the shape memory material may be configured to change a state of the self-retaining retractor between a collapsed state and an expanded state. Accordingly, in the collapsed state, the self-retaining retractor may be conveniently inserted into the orifice. Subsequently, the self-retaining retractor may be changed to the expanded state in which the self-retaining retractor applies outwardly radial force causing the orifice to increase in size.

### RELATED APPLICATION

Under provisions of 35 U.S.C. §119(e), the Applicant claims the benefit of U.S. provisional application No. 62/311,245, filed on Mar. 21, 2017, which is incorporated herein by reference.

It is intended that the referenced application may be applicable to the concepts and embodiments disclosed herein, even if such concepts and embodiments are disclosed in the referenced application with different limitations and configurations and described using different examples and terminology.

### FIELD OF DISCLOSURE

The present disclosure generally relates to flexible retractors. More specifically, the present disclosure relates to self-retaining radial tissue flexible retractors.

### BACKGROUND

Retractors are generally used by medical practitioners to expand an orifice in a body for improved visibility and/or access into the orifice. For instance, surgical retractors are commonly used to hold tissue away from an operative area to improve exposure during surgery. Such retractors are typically inserted within an incision during surgery to forcibly enlarge the area of incision and facilitate access within the orifice.

Conventional hand-held and weighted retractors most often take the form of spatulate hooks. Using these retractors, it may be necessary to exert traction from the outside by an operating surgeon or an assistant. Often multiple retractors may be required and accordingly multiple assistants to operate them. This may lead to crowding of a limited operating space.

On the other hand, self-retaining retractors reduce the need for additional hands in the operating space. Self-retaining retractors often take the form of pivoting blades that lock in place with traction from the tissues being retracted. These are often difficult to place and reposition, and may deform the orifice in undesirable ways.

Further, a problem with conventional retractors, such as rigid metal retractors, is that there is a risk of injury to the surrounding delicate soft tissue due to compression.

Accordingly, there is a need for improved retractors that may over come at least some of the problems with current retractors.

### BRIEF OVERVIEW

A self-retaining radial tissue retractor may be provided. This brief overview is provided to introduce a selection of concepts in a simplified form that are further described below in the Detailed Description. This brief overview is not intended to identify key features or essential features of the claimed subject matter. Nor is this brief overview intended to be used to limit the claimed subject matter's scope.

The present disclosure describes a self-retaining retractor comprising a moderately flexible structure that may be compressed for insertion into an orifice, such as, for example, the vagina. Upon placement, the self-retaining retractor may be expanded manually and/or automatically. Accordingly, the self-retaining retractor may utilize flexible or pre-set shape memory materials. As a result of the expansion, radial forces may be applied on the walls of the orifice, stretching tissues and increasing the size of the orifice. Further, the self-retaining retractor, in an expanded state, may have a shape of a tube, though modified for specific anatomy. The shape may be designed for specific anatomy, such as, for example, the vagina. Accordingly, the diameter of the tube may change along the length so as to more properly fit and reduce movement of the self-retaining retractor with respect to surrounding tissues. Additionally, the self-retaining retractor may be of a low profile such that the field of view is maximized. Furthermore, the self-retaining retractor may be flexible enough to allow the use of spatula based retractors on top of the self-retaining retractor. For example, temporary access to certain tissues may require additional retraction to one side.

In some embodiments, the self-retaining retractor may include a wire made of a material, such as, but not limited to, one or more of nitinol, a metal and a polymer. Further, the wire may be formed in such a shape that allows for a collapsible structure, such as a tubular form. Accordingly, the self-retaining retractor may be inserted into an orifice in a collapsed state. Further, once in place inside the orifice, the self-retaining retractor may be expanded in order to increase the size of the orifice. In some embodiments, the collapsible structure may be based on one or more of a bucket handle configuration, a cylindrical clam shell configuration and a scissoring configuration. Further, the collapsible structure may have the ability to change profile, such as, the diameter, during different stages such as insertion, retraction and removal of the self-retaining retractor. Additionally, the collapsible structure may be configured to be opened manually and/or with the aid of custom tools, and may be secured in an open position until removal is desired.

Both the foregoing brief overview and the following detailed description provide examples and are explanatory only. Accordingly, the foregoing brief overview and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the Applicant. The Applicant retains and reserves all rights in its trademarks and copyrights included herein, and grants permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure. In the drawings:

FIG. 1 illustrates a bucket handle configuration of a self-retaining retractor in a collapsed state and an expanded state consistent with the present disclosure;

FIG. 2 illustrates a cylindrical clam shell configuration of a self-retaining retractor in a collapsed state and an expanded state consistent with the present disclosure;

FIG. 3 illustrates a tubular scissor configuration of a self-retaining retractor in a collapsed state and an expanded state consistent with the present disclosure;

FIG. 4 illustrates a self-retaining retractor of a conical shape in a collapsed state and an expanded state consistent with the present disclosure;

FIG. 5 illustrates a self-retaining retractor made of a wound polymer sheet consistent with the present disclosure;

FIG. 6 illustrates a self-retaining retractor made of a wound polymer sheet with nesting of the overlapping layers consistent with the present disclosure;

FIG. 7 illustrates a ratcheting mechanism of a self-retaining retractor made of a sheet of a material such as a polymer in a wound state consistent with the present disclosure;

FIG. 8 illustrates a ratcheting mechanism of a self-retaining retractor made of a sheet of a material such as a polymer in an unwound state consistent with the present disclosure;

FIG. 9 illustrates a catch and release mechanism of a self-retaining retractor made of a wound polymer sheet consistent with the present disclosure;

FIG. 10 illustrates a beveled catch and release mechanism of a self-retaining retractor made of a wound polymer sheet consistent with the present disclosure;

FIG. 11 illustrates a removable release mechanism of a self-retaining retractor consistent with the present disclosure; and

FIG. 12 illustrates use of a self-retaining retractor for holding stay sutures consistent with the present disclosure.

### DETAILED DESCRIPTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Regarding applicability of 35 U.S.C. §112, ¶6, no claim element is intended to be read in accordance with this statutory provision unless the explicit phrase “means for” or “step for” is actually used in such claim element, whereupon this statutory provision is intended to apply in the interpretation of such claim element.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header.

The present disclosure includes many aspects and features. Moreover, while many aspects and features relate to, and are described in, the context of vaginal use, embodiments of the present disclosure are not limited to use only in this context.

I. Overview

Consistent with embodiments of the present disclosure, a self-retaining radial tissue retractor may be provided. This overview is provided to introduce a selection of concepts in a simplified form that are further described below. This overview is not intended to identify key features or essential features of the claimed subject matter. Nor is this overview intended to be used to limit the claimed subject matter's scope. The self-retaining radial tissue retractor may be used by individuals, such as medical practitioners, or organizations, such as hospitals, to expand an orifice of a body for improved visibility and/or access to the orifice such as, for example, the vaginal orifice.

The present disclosure relates generally to retractors, such as for example, surgical retractors. More specifically, the present disclosure relates to an improved retractor comprised of flexible material that can be compressed and inserted into a small orifice, then expanded to increase the size of said orifice. The orifice may be a natural orifice such as, for example, the vaginal orifice. Alternatively, the orifice may be a result of an incision made on the skin during surgery.

The self-retaining retractor of the present disclosure may be designed to expand and exert radial forces when placed within the orifice. As a result, the self-retaining retractor may provide a greater view inside the orifice and more accessibility to underlying tissue. The self-retaining retractor may be constructed of a suitable material, such as, for example, a shape memory material that may be configured to change between and/or maintain at the self-retaining retractor an expanded state and a collapsed state. For example, the self-retaining retractor may be used to expand a surgical incision at an operative site below skin surface by applying force radially outward and holding tissue back using the radial pressure. Further, the self-retaining retractor may be designed to remain in place until removal.

In general, the self-retaining retractor may be designed to be maintained in one of the collapsed state and the expanded state. In the collapsed state, the dimensions of the self-retaining retractor may be such that, insertion into an orifice may be facilitated. For example, in the collapsed state, a diameter of a tubular self-retaining retractor may be smaller. Further, in the expanded state, the dimensions of the self-retaining retractor may be such that, at least one portion of the self-retaining retractor may be compressed towards at least one part of tissue proximal to the orifice. For example, in the expanded state, a diameter of a tubular self-retaining retractor may be larger resulting in external wall of the tubular self-retaining retractor to be pushed against the inner wall of an orifice, such as the vaginal orifice.

Further, in the expanded state, the self-retaining retractor may be designed to remain in the expanded state until a force is exerted to change the self-retaining retractor to the collapsed state. In other words, the self-retaining retractor may be configured to remain in the expanded state in the absence of any altering forces. Accordingly, there may be no need for a medical practitioner or a medical assistant to manually hold the self-retaining retractor.

Both the foregoing overview and the following detailed description provide examples and are explanatory only. Accordingly, the foregoing overview and the following detailed description should not be considered to be restrictive. Further, features or variations may be provided in addition to those set forth herein. For example, embodiments may be directed to various feature combinations and sub-combinations described in the detailed description.

II. Configuration

In general, the self-retaining retractor may be configured to change between the collapsed state and the expanded state based on one or more factors such as, an applied force, pressure, temperature, ph value, contact with tissue etc.

For example, in some embodiments, a force may be required to be manually applied in order to set the self-retaining retractor in the collapsed state. For example, the force may be applied using custom tools adapted to conform to an inner profile of the self-retaining retractor. Similarly, in some other embodiments, a force may be required to be manually applied in order to set the self-retaining retractor in the expanded state.

Further, in some embodiments, use of shape memory materials may determine a natural state of the self-retaining retractor to be one of the expanded state and the collapsed state. Further, a force may be required to be applied in order to change the natural state of the self-retaining retractor.

As another example, the self-retaining retractor may automatically change to the expanded state when in contact with tissue. Accordingly, the self-retaining retractor may include temperature sensitive materials that are configured to alter shape on being exposed to normal body temperatures.

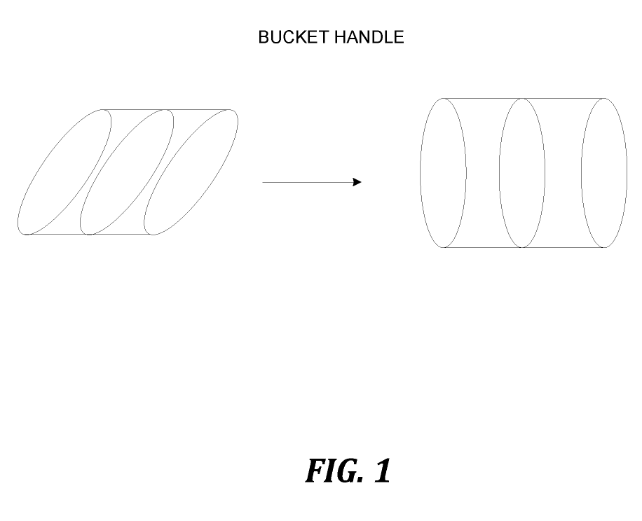

FIG. 1 illustrates a bucket handle configuration of a self-retaining retractor in a collapsed state and an expanded state consistent with the present disclosure. As shown, the self-retaining retractor may be in the form of a tubular structure. In some embodiments, the tubular structure may be formed out of an elastic polymer sheet interspersed with rings of shape memory materials such as nitinol wires.

Further, a profile of the tubular structure, such as the diameter may be smaller in size in the collapsed state. Accordingly, insertion of the self-retaining retractor into an orifice, such as the vaginal orifice, may be facilitated. Further, as shown, after being placed within the orifice, the self-retaining retractor may change to the expanded state. For example, the diameter of the tubular structure may increase thus applying radial forces towards the periphery of the orifice. As a result, a diameter of the orifice may increase provided greater view and accessibility into the orifice.

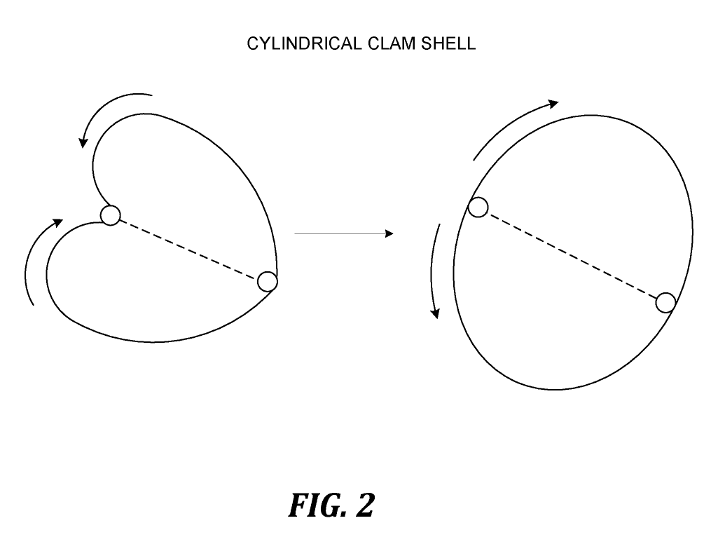

FIG. 2 illustrates a cylindrical clam shell configuration of a self-retaining retractor in a collapsed state and an expanded state consistent with the present disclosure. For instance, the cylindrical clam shell configuration may include an elastic polymer sheet in the form of a tubular structure with a clam shell shaped cross-sectional profile.

Further, shape memory materials, such as a nitinol wire, may be incorporated within the elastic polymer, for example, between two nodes as illustrated. Accordingly, in the collapsed state, the length of the nitinol wire may be shorter. As a result, in the collapsed state, a cross sectional area of the self-retaining retractor may be smaller allowing for easier insertion into the orifice.

However, subsequent to placement into the orifice, the self-retaining retractor may be changed to the expanded state based on an applied force and/or automatically due to the presence of shape change materials incorporated within the self-retaining retractor. For example, the length of the nitinol wire may increase causing the self-retaining retractor to expand along the cross-section.

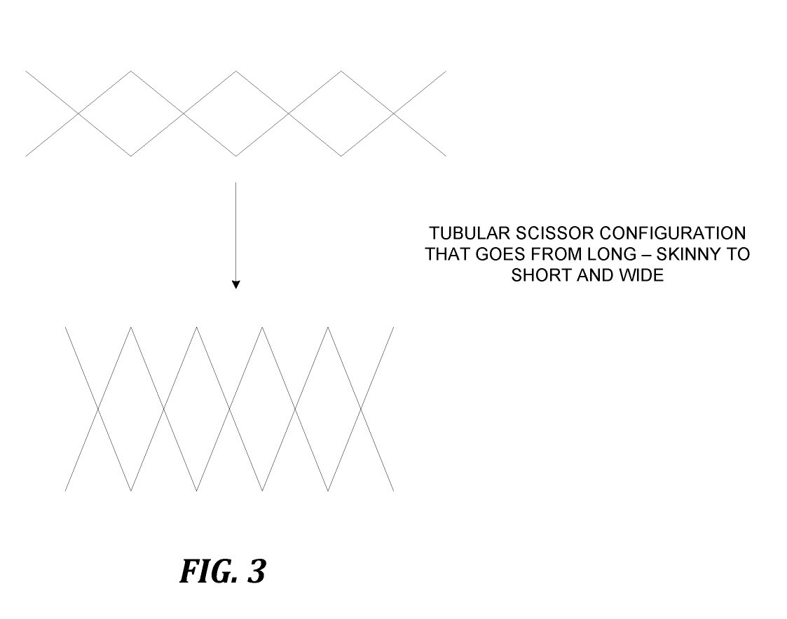

FIG. 3 illustrates a tubular scissor configuration of a self-retaining retractor in a collapsed state and an expanded state consistent with the present disclosure. In some embodiments, the tubular scissor configuration may include a mesh made of shape change material, such as nitinol, in the form of wires.

Further, in the collapsed state, a profile of the self-retaining retractor may be long and of a smaller cross-sectional area. Accordingly, insertion of the self-retaining retractor may be facilitated.

However, subsequent to insertion into the orifice, the self-retaining retractor may be changed to the expanded state with a profile of the self-retaining retractor being shorter and wider. Accordingly, a cross-sectional area of the self-retaining retractor may be greater in the expanded state causing radial forces to be applied along the periphery and/or walls of the orifice resulting in increased size of the orifice.

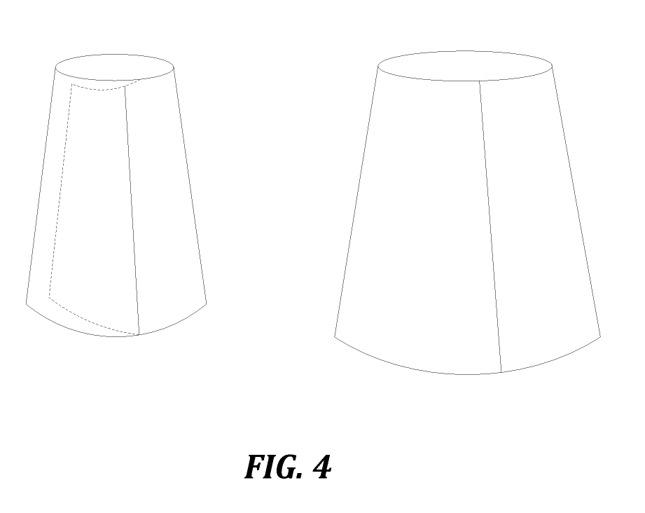

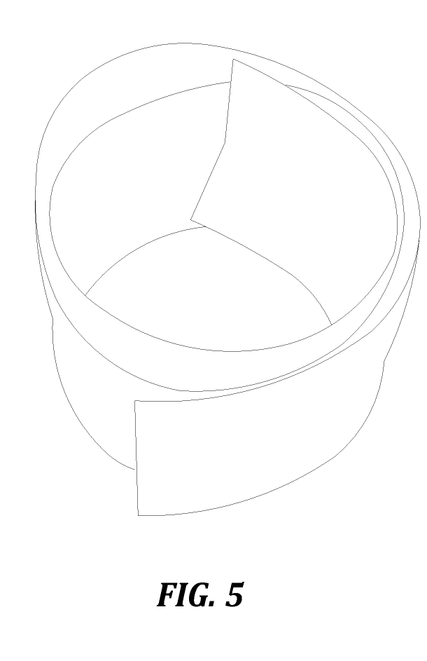

FIG. 4 illustrates a self-retaining retractor of a conical shape in a collapsed state and an expanded state consistent with the present disclosure. Accordingly, the self-retaining retractor may include a flexible and elastic polymer sheet or mesh. Such an elastic polymer may be contracted and held for delivery by overlapping and winding the walls of the elastic polymer as illustrated in FIG. 5 such that an outward force may be exerted when released.

A shape of the self-retaining retractor may be, for example, cylindrical, conical, or other similar shape that conforms to specific body anatomy. Accordingly, in the collapsed state, the self-retaining retractor may be in the form of a conical structure with a smaller cross-sectional area and/or volume. Further, as illustrated in FIG. 4, in the expanded state, the self-retaining retractor may be of a larger cross-sectional area and/or volume causing expansion of the orifice.

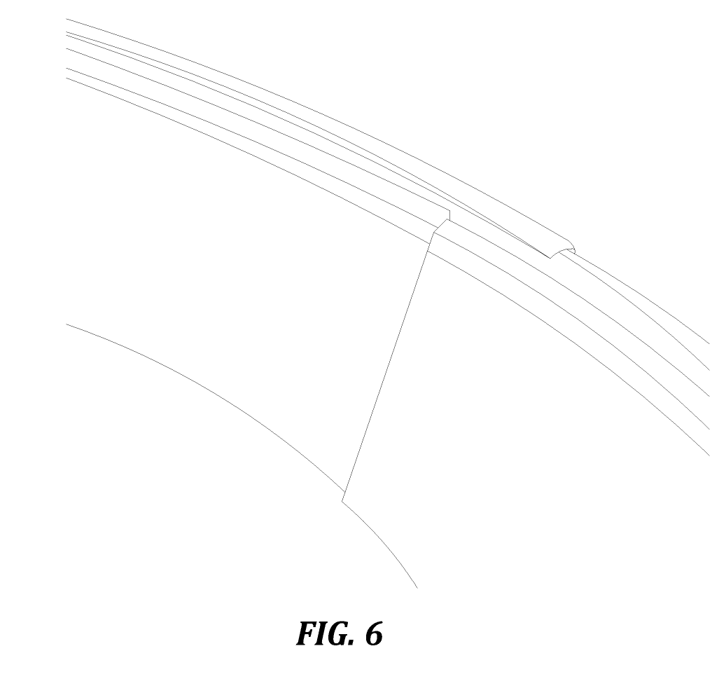

FIG. 6 illustrates a self-retaining retractor made of a wound polymer sheet with nesting of the overlapping layers consistent with the present disclosure. In this embodiment, a curled or ridged form factor may be incorporated at one or both edges in order to keep the overlapping layers from sliding axially in or out. Such a curl would be along the edge and each successive layer would be nesting in the previous layer. As the self-retaining retractor expands or contracts, this curled form factor will keep the layers aligned. An example of a similar design is in a spring form pan that changes diameter with overlapping layers, but the layers remain connected along one edge as they slide over each other.

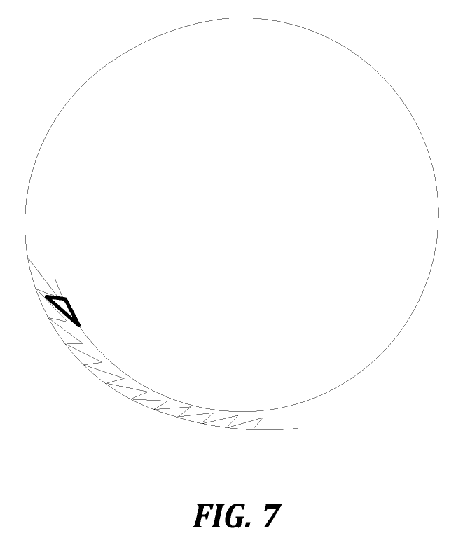

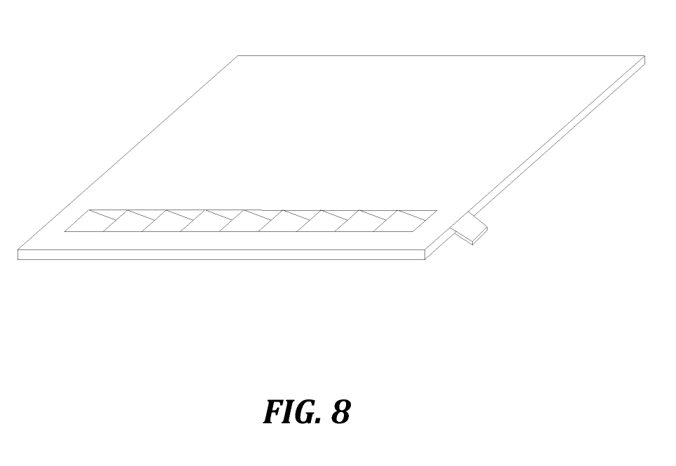

FIG. 7 and FIG. 8 illustrates a ratcheting mechanism of a self-retaining retractor made of a polymer sheet in a wound state and unwound state respectively, consistent with the present disclosure. The ratcheting mechanism may be configured to temporarily lock the self-retaining retractor in place (i.e. does not allow a decrease in diameter) after being set by an operator. This may be similar to the locking mechanism on commercial zip-ties or other one-way locking mechanisms. Such a mechanism would have a plurality of saw-tooth ridges (with one sloping and one steep side) such that a corresponding catch, or catches, would easily move along the sloping angles, but not be able to travel backwards across the steeper side. These ridges may be recessed in order to lower profile and reduce chances of catching on tissue. One or more release mechanisms such as those described below may be used to release retractor from its expanded state into a smaller diameter to ease removal.

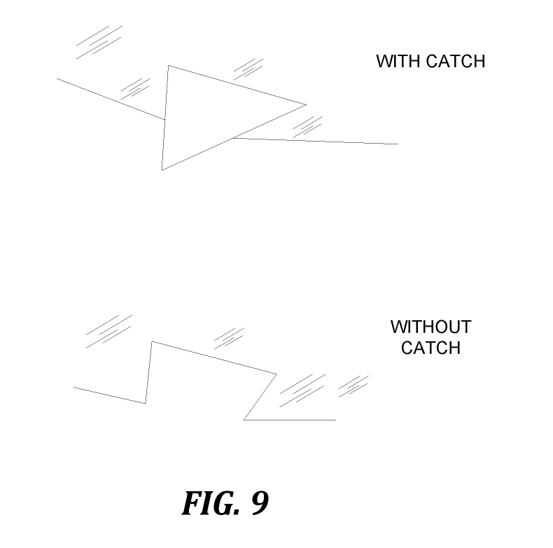

FIG. 9 illustrates a catch and release mechanism of a self-retaining retractor made of a wound polymer sheet consistent with the present disclosure. The release mechanism may be operated by means of removing or blocking ratchet catch as shown. Accordingly, the release mechanism may be included to disengage the ratchet components when desired. This release mechanism may include at least one removable catch component so that when retraction is no longer desired, the catch can be pulled out or moved out of position. This component may be held in place by specific shape configurations, such as dovetail and socket forms conventional to carpentry as illustrated.

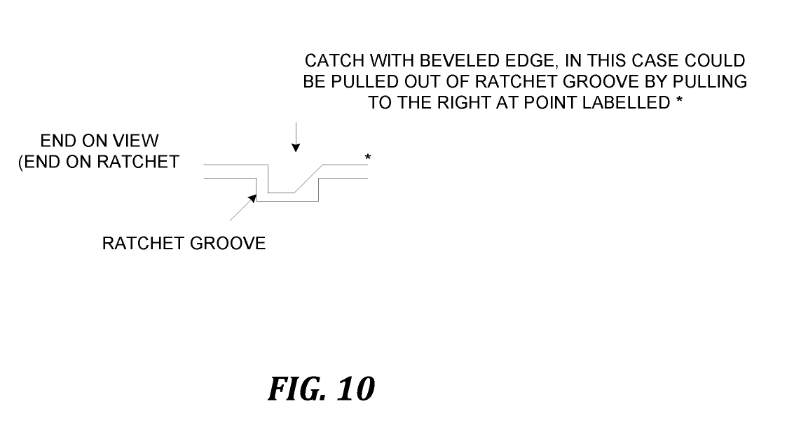

FIG. 10 illustrates a beveled catch and release mechanism of a self-retaining retractor made of a wound polymer sheet consistent with the present disclosure. In this embodiment, the release mechanism may be operated by pulling on a designated corner, labelled as*in FIG. 10. Accordingly, in this release mechanism, the catch on the ratchet may engage with the ridges during expansion and hold for the duration of desired retraction. When desired, an operator may pull on or near the inner corner. As illustrated, the catch may include a bevel (angled) edge such that lateral traction applied during this motion is sufficient to pull the catch out of the recessed channel containing the ridges (ratchet groove). This motion has the double effect of disengaging the ratchet mechanism and reducing the diameter of the self-retaining retractor to allow for removal from the orifice. Alternatively or additionally, the ratchet groove may have a beveled edge to allow lateral force to disengage (not shown in the figures).

Further, in some embodiments, to allow for different diameters at different depths into the body, more than one of these mechanisms may be used at the same time. More specifically, multiple separate ratchet tracks, each of a different type, may be used to give close control over device shape when inserted into the body.

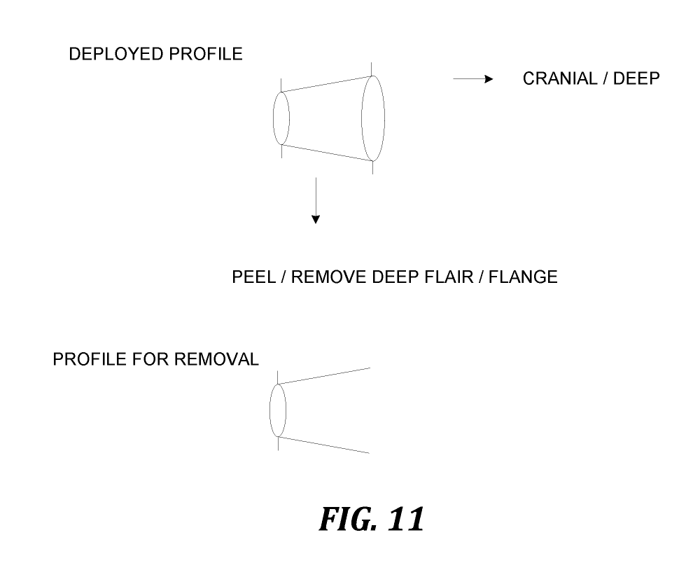

FIG. 11 illustrates a removable release mechanism of a self-retaining retractor consistent with the present disclosure. The removable release mechanism may include a removable portion of the self-retaining retractor. The removable portion may be of a shape profile so as to keep the self-retaining retractor from moving out, such as a flange at the distal end. Upon completion of use, the removable portion may be removed by pulling it apart from the main body of the self-retaining retractor. Further, the removable portion may only be attached by a thinner portion, similar to perforated lines to ease tearing of paper or other thin connector that allows for easy removal. Once the removable portion has been removed, the shape of the self-retaining retractor may allow for it to slide out of the body.

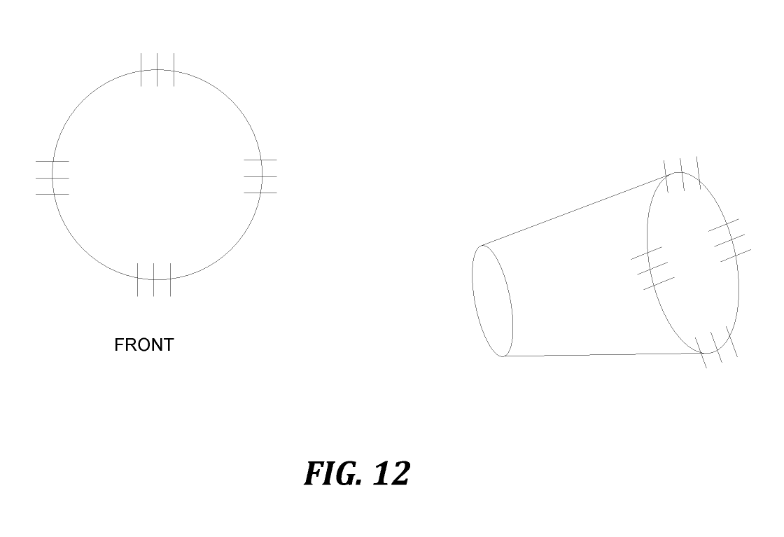

FIG. 12 illustrates use of a self-retaining retractor for holding stay sutures consistent with the present disclosure. During vaginal hysterectomies, a surgeon may place stay sutures through the ligaments holding up the cervix. As the structures are cut, sutures are placed to gain traction on the ligament as they dissect them away from the cervix. Slats can be placed into the self-retaining retractor to hold the stay sutures. Accordingly, the self-retaining retractor's ability to hold the stay sutures may free the surgeon from holding these sutures. These slats may be radially distributed along edge of the self-retaining retractor as shown.

Further, in some embodiments, an additional feature that may be included is a textured outer wall. This texture may be added to increase traction on tissue being retracted and thus minimize movement of the self-retaining retractor.

Additionally, the inside and/or outside walls of the self-retaining retractor may be coated in such a way as to minimize friction between tissue and the self-retaining retractor. Such lubricious walls on the interior surface of the self-retaining retractor may ease, for example, removal of uterus. Further, such coating on the outer walls may ease removal of the self-retaining retractor from the orifice.

Further, in some embodiments, the self-retaining retractor may be constructed from material that may be cut resistant, thus protecting tissue walls from accidental cuts. Alternatively, the self-retaining retractor may be made up of multiple layers with the innermost layer being cut resistant.

Furthermore, in some embodiments, the self-retaining retractor may also incorporate force indication to communicate the amount of force applied to the tissue. Accordingly, an operator may be warned of excessive force being applied to the tissue. Thus, the force indication may aid in reducing tissue injury.

Additionally, in some embodiments, the self-retaining retractor may also include illumination. Accordingly, one or more fiber optic component may be provided and be illuminated by an external or integrated light source, such as a battery powered LED or light guide cable (such as fiber optic) to transmit light from an external light source.

III. Technical Advantages

The following is a non-exhaustive list of some technical advantages of the embodiments presented herein.

Conventional technology may rely solely on the self-expanding nature of the material for retractor strength. The present disclosure presents embodiments that contain a complete tube shape that is mechanically stronger and relies more on compressive strength rather than bending strength.

Conventional technology may require a drive structure to expand, where the present disclosure presents embodiments that are combine self-expanding and applied pressure to reach an expanded state.

Conventional technology may comprise an elongated sleeve that has adjustable length, rather than the diameter of the opening. Such conventional technology may also rely on its pre-set elastic strength to provide retraction.

IV. Claims

While the specification includes examples, the disclosure's scope is indicated by the following claims. Furthermore, while the specification has been described in language specific to structural features and/or methodological acts, the claims are not limited to the features or acts described above. Rather, the specific features and acts described above are disclosed as example for embodiments of the disclosure.

Insofar as the description above and the accompanying drawing disclose any additional subject matter that is not within the scope of the claims below, the disclosures are not dedicated to the public and the right to file one or more applications to claims such additional disclosures is reserved.

The following is claimed:

1. A self-retaining retractor configured for insertion into an orifice, the self-retaining retractor comprising:
a flexible structure; and
an expandable material that is configured to change a state of the self-retaining retractor between a collapsed state and an expandable state.

2. The self-retaining retractor of claim 1, wherein the flexible structure comprises at least one of an elastic polymer sheet and an elastic polymer mesh.

3. The self-retaining retractor of claim 1, wherein the self-retaining retractor is configured to exert a radial force towards the periphery of the orifice in the expanded state.

4. The self-retaining retractor of claim 1, wherein the shape memory material is incorporated in the flexible structure in at least one of a bucket handle configuration, a cylindrical clam shell configuration and a scissoring configuration.

5. The self-retaining retractor of claim 1, wherein a shape of the self-retaining retractor is at least one of a cylindrical form and a conical form.

6. The self-retaining retractor of claim 1, wherein at least one edge of the self-retaining retractor comprises a ridged form factor configured to maintain overlapping layers of the self-retaining retractor from sliding axially.

7. The self-retaining retractor of claim 1 further comprising at least one ratcheting mechanism configured to temporarily lock the self-retaining retractor in a desired state.

8. The self-retaining retractor of claim 1 further comprising a release mechanism configured to release the self-retaining retractor from the desired state.

9. The self-retaining retractor of claim 1 configured to allow the use of a spatula based retractor on top of the self-retaining retractor.

10. The self-retaining retractor of claim 1, wherein the expandable material comprises a shape memory material incorporated in the flexible structure, wherein the shape memory material is configured to change the state of the self-retaining retractor between the collapsed state and the expanded state.

11. The self-retaining retractor of claim 1, further comprising a structure to control the depth into the body that the retractor will sit.
